(19) United States
(12) Patent Application Publication
(10) Pub. No.: US 2010/0044673 A1
(43) Pub. Date: Feb. 25, 2010

(54) LABELING FLUORESCENT COMPOUND

(75) Inventors: Kazuya Tsukada, Kanagawa (JP); Kazuyoshi Goan, Kanagawa (JP); Hisatake Okada, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA MEDICAL & GRAPHIC, INC., Tokyo (JP)

(21) Appl. No.: 12/531,092

(22) PCT Filed: Mar. 26, 2008
(86) PCT No.: PCT/JP2008/055682

§ 371 Date: Sep. 14, 2009

(30) Foreign Application Priority Data
Mar. 29, 2007 (JP) 2007087254

(51) Int. Cl. H01L 33/00; G01N 33/58; C09K 11/08

(52) U.S. Cl. 257/13; 428/403; 977/773; 257/E33.005

(57) ABSTRACT

A labeling fluorescent compound which enables highly stable detection in vital labeling and has high sensitivity. The labeling fluorescent compound is characterized by being composed of inorganic fluorescent nanoparticles which have a surface modification compound disposed on the surface thereof and have an average particle diameter of 1.0-20 nm. It is further characterized in that the proportion of the length of the surface modification compound as measured from the surface of the inorganic fluorescent nanoparticles to the particle diameter of the inorganic fluorescent nanoparticles is from 0.10 to 0.50, and that the proportion of the specific gravity of the inorganic fluorescent nanoparticles having the surface modification compound fixed thereto to the specific gravity of the inorganic fluorescent nanoparticles not having the surface modification compound is from 0.80 to 0.40.

### TECHNICAL FIELD

The present invention relates to a labeling fluorescent compound, and more specifically to a labeling fluorescent compound used for basic research in molecular imaging.

### BACKGROUND

Basic research in molecular imaging is actively being conducted in order to clarify molecular dynamics, intermolecular interactions, and molecular position information via visual imaging applied to living body molecules as targets in which living cells or small animals are used as subjects and thereby to reveal life science mechanisms and make drug discovery. Conventionally, organic colorants and organic fluorescent proteins, which are considered to exhibit high fluorescent sensitivity, have mainly been used for labeling. However, adequate sensitivity is unrealized. Thereby, when excitation light intensity is intended to be increased for detection sensitivity enhancement, there have been produced problems of phototoxic properties resulting in invasive properties to living body molecules and of poor stability due to photodecomposition. Therefore, a wide variety of researches and investigations on inorganic fluorescent materials exhibiting enhanced stability and high sensitivity are being conducted.

Conventional labeling inorganic fluorescent materials emitting detection light employing inorganic fluorescent materials are composed of a core or core/shell particle and a surface modifier. To disperse such a surface modifier in an aqueous medium corresponding to vital conditions and to realize specific adsorption thereof to a target living body molecule, various types of surface modifiers are being used (for example, refer to Patent Document 1).

In such conventional labeling, non-uniform aggregation or association is made, resulting in the problems that detection sensitivity for a target molecule varies and the accuracy of authenticity in living body molecule detection decreases.

Patent Document 1: Japanese Patent Publication open to Public Inspection No. 2006-70249

### DISCLOSURE OF THE INVENTION

### Problems to be Solved by the Invention

An object of the present invention is to provide a labeling fluorescent compound exhibiting extremely stable detection performance in vital labeling with high sensitivity.

### Means to Solve the Problems

The above object of the present invention can be achieved by the following constitutions.

1. A labeling fluorescent compound comprising an inorganic fluorescent nanoparticle which is provided with a surface modifying compound on a surface of the inorganic fluorescent nanoparticle, provide that the inorganic fluorescent nanoparticle has an average particle diameter of 1.0-20 nm, wherein a ratio of a length of the surface modifying compound from the surface of the inorganic fluorescent nanoparticle to a particle diameter of the inorganic fluorescent nanoparticle is 0.10-0.50; and a ratio of a specific gravity of the inorganic fluorescent nanoparticle bonded with the surface modifying compound to a specific gravity of an inorganic fluorescent nanoparticle non-bonded with the surface modifying compound is 0.80-0.40.

2. The labeling fluorescent compound described in item 1, wherein the inorganic fluorescent nanoparticle has a core/shell structure composed of a semiconductor nanocrystal core and a semiconductor shell.

3. The labeling fluorescent compound described in items 1 or 2, wherein the inorganic fluorescent nanoparticle emits infrared radiation by excitation with rays in the range of near-infrared radiation to infrared radiation.

### EFFECTS OF THE INVENTION

The present invention has enabled to provide a labeling fluorescent compound exhibiting extremely stable detection performance in vital labeling with high sensitivity.

### DESCRIPTION OF THE PREFERRED EMBODIMENT

The present invention will now be detailed.

The length of the surface modifying compound of the present invention is 0.10-0.50 times as long as the average particle diameter of an inorganic fluorescent nanoparticle, preferably 0.15-0.4 times, most preferably 0.2-0.35 times.

The length of such a surface modifying compound can be determined by any well known method. For example, the average particle diameter of a labeling fluorescent compound bonded with a surface modifying compound is determined using a ZETASIZER (produced by Sysmex Corp.), and then the above length can be calculated from the average particle diameter of an inorganic fluorescent nanoparticle non-bonded with the surface modifying compound having been previously determined via the same method.

The surface modifying compound of the present invention is a compound having a group which is adsorbed to the surface of an inorganic fluorescent nanoparticle and a group which is bonded or adsorbed to a living body molecule. Such a group being adsorbed to the surface of an inorganic fluorescent nanoparticle includes a mercapto group, an amino group, phosphonic acid, sulfonic acid, and a silicate compound. Of these, any appropriate one can be selected depending on the composition of an inorganic fluorescent material. As the group being bonded or adsorbed to a living body molecule, those applicable to amino acids are suitable, including a carboxyl group and an amino group.

As specific surface modifying compounds, for example, the following compounds can be listed, but the present invention is not limited to these compounds.

A: Mercaptoacetic acid

B: 2-Mercaptopropionic acid

C: 3-Mercaptopropionic acid

D: 2-Mercaptobutyric acid

E: 4-Mercaptobutyric acid

F: 8-Mercaptooctanoic acid

G: 11-Mercaptoundecanoic acid

H: 11-Mercaptododecanoic acid

Length of a surface modifying compound can be changed via various methods and is adjusted to fall within the range of the present invention according to the average particle diameter of an inorganic fluorescent nanoparticle. For example, the distance between a group being adsorbed to the surface of an inorganic fluorescent nanoparticle and a group being bonded or adsorbed to a living body molecule is adjusted by the length of an alkyl chain or an ethylene glycol chain (—(CH₂CH₂O)ₓ—). Further, the specific gravity of an inorganic fluorescent nanoparticle bonded with a surface modifying compound is 0.80-0.40 times as large as the specific gravity of an inorganic fluorescent nanoparticle non-bonded with the surface modifying compound, preferably 0.80-0.45 times, most preferably 0.60-0.50 times.

Such a specific gravity specified by the present invention can be determined by an Archimedes method utilizing buoyancy. Using a liquid having a known specific gravity, buoyancy F=A−B is determined from weighed value A of a solid in the air and weighed value B of the solid in the liquid and then the specific gravity is determined by the density of the solid ρ=(A/F)×ρo, provided that the density of the liquid is designated as ρo. The same determination can be conducted by designating this solid as an inorganic fluorescent nanoparticle or an inorganic fluorescent nanoparticle adsorbed with a surface modifying compound.

The average particle diameter of the inorganic fluorescent nanoparticle of the present invention is 1.0-20 nm, preferably 1.0-10 nm. The average particle diameters of an inorganic fluorescent nanoparticle bonded with a surface modifying compound and an inorganic fluorescent nanoparticle bonded with no surface modifying compound can be determined by a well known method, for example, using a ZETASIZER (produced by Sysmex Corp.).

The inorganic fluorescent nanoparticle of the present invention preferably features a core/shell structure. The band gap of the shell is preferably higher than that of the core. The shell is needed to stabilize surface defects of core particles and to enhance luminance, and is also important to form a surface to which a surface modifying compound is easily adsorbed or bonded. Further, to produce the effects of the present invention, such a structure became critical to enhance the accuracy of detection sensitivity.

The inorganic fluorescent nanoparticle of the present invention is a semiconductor nanoparticle having a core particle diameter enabling to emit infrared radiation via excitation using radiation such as near-infrared radiation (about 700 nm-1300 nm). This can be realized by adjusting the particle diameter to fall within the infrared range by use of a quantum size effect. A shell is prepared in such a manner that surface defects are stabilized to sufficiently display fluorescence emitted by core particles.

Further, in the labeling of the present invention, infrared emission via near-infrared-infrared excitation is desirable from the viewpoint of non-invasive properties to living body molecules. In the case of small animal imaging, from the viewpoint of transparency of living tissues due to wavelengths in the infrared ranger detection with high sensitivity can be realized in a deeper in vivo area, which is significantly useful. Accordingly, the constitution of the present invention is specifically suitable.

In an HF etching method to be described later, inorganic fluorescent nanoparticles of Si (hereinafter referred to also as “Si semiconductor fine particles”, “Si fine particles”, or “Si core particles”) can allow Si fine particles of different size to be precipitated by adjusting annealing duration. Further, in an anodization method, Si fine particles of different size can be obtained by varying energization duration.

Inorganic fluorescent nanoparticles include inorganic fluorescent materials and semiconductor nanoparticles employing an activator. As the semiconductor nanoparticles, quantum dots exerting a quantum effect are preferably used.

The quantum dots refer to particles wherein when inorganic semiconductor particles are turned into nano-level particle diameter sizes (depending on the composition), a quantum confinement effect is expressed and excitation energy is conserved, whereby conversion to fluorescence is realized with high efficiency, resulting in enhanced emission intensity and in obtaining different emission wavelengths by a quantum size effect when the sizes of the particles are changed. Accordingly, use as a labeling agent realizes high detection performance and high stability, compared to colorants.

As the quantum dots, those in the IV group of the periodic table of the elements are used. Of these, Si or Ge is preferably used.

The inorganic fluorescent nanoparticle of the present invention emits fluorescence in the range of 350 nm-1100 nm. Near-infrared emission is preferably employed to inhibit emission effects possessed by living cells themselves and then enhance an SN ratio.

The semiconductor nanoparticle of the present invention may have a core composition and a shell composition to cover the surface thereof. When the semiconductor nanoparticle is a quantum dot, defects on the core surface is subjected to passivation treatment via shell formation, whereby quantum efficiency can be enhanced. SiO₂ and GeO₂ can be used for Si and Ge, respectively. However, there is no limitation and ZnS can also be used for such a shell.

The particle diameter of an inorganic fluorescent nanoparticle prior to surface modification is 1-20 nm, preferably 1-10 nm. In the case of more than 20 nm, application to cell imaging as a labeling compound is difficult, resulting in a narrower application range. In contrast, in the case of less than 1 nm, the balance with surface modification is difficult to achieve, leading to emission inhibition.

### Examples

### Preparation of Si Core Particles and Si/SiO₂.Core/Shell Particles

(HF Etching Method)

When inorganic fluorescent nanoparticles of Si are produced by dissolving heat-treated SiOₓ (x: 1.999) in hydrofluoric acid, initially, SiOₓ (x: 1.999) coated on a silicon wafer via plasma CVD is annealed under an ambience of an inert gas at 1100° C. Thereby, Si semiconductor fine particles (crystals) are precipitated in the SiO₂ film.

Subsequently, this silicon wafer is treated with a hydrofluoric acid aqueous solution of about 1% at room temperature to remove the SiO₂ film, and then Si semiconductor fine particles of a size of several nm having been aggregated on the liquid surface are recovered. Herein, via this hydrofluoric acid treatment, the dangling bonds (unattached hands) of the Si atoms on the semiconductor fine particle (crystal) surface are subjected to hydrogen termination, resulting in stabilized Si crystals. Thereafter, the surface of the thus-recovered Si semiconductor fine particles is heat-oxidized by heating under an ambience of oxygen at 800-1000° C. for 1.5 hours and then a shell layer composed of SiO₂ is formed in the circumference of a core composed of the Si semiconductor fine particles. The average particle diameter of inorganic fluorescent nanoparticles composed of this Si/SiO₂.core/shell was determined using a ZETASIZER (produced by Sysmex Corp.) and the results were listed in Table 1.

(Anodization Method)

Further, when Si semiconductor fine particles are produced via anodization of a p-type silicon wafer, initially, Si semiconductor fine particles (crystals) are precipitated in such a manner that a p-type silicon wafer and platinum are arranged as opposed electrodes in a solution prepared by mixing hydrofluoric acid (46%), methanol (100%), and hydrogen peroxide solution (30%) at a ratio of 1:2:2 and energization of 320 mA/cm² is carried out at 25° C. for 20 minutes. The surface of the thus-obtained Si semiconductor fine particles is heat-oxidized under an ambience of oxygen at 500-650° C. for 1.5 hours and then a shell layer composed of SiO₂ is formed in the circumference of a core composed of the Si crystals. The average particle diameter of inorganic fluorescent nanoparticles composed of this Si/SiO₂.core/shell was determined using a ZETASIZER (produced by Sysmex Corp.) and the results were listed in Table 1.

(Preparation of Si/ZnS.Core/Shell Particles)

The above-obtained Si core particles are dispersed in pyridine and kept warm at 100° C. Separately, Zn(C₂H₅)₂, ((CH₃)₃Si)₂S and P(C₄H₉)₃ were slowly mixed under an ambience of argon gas at 100° C. while applied with ultrasound.

The resulting mixture is added to the pyridine dispersion by dripping. After addition, the temperature was controlled at 100° C. and the pH (8.0) was kept constant, followed by slow stirring for 30 minutes. The resulting product was centrifuged and then precipitated particles were collected. Via elemental analysis of the thus-obtained particles, Si and ZnS were verified. Then, via XPS analysis, it was verified that the Si surface was covered with the ZnS. The average particle diameter of inorganic fluorescent nanoparticles composed of this Si/ZnS.core/shell was determined using a ZETASIZER (produced by Sysmex Corp.) and the results were listed in Table 1.

(Introduction of a Surface Modifying Compound)

When a living substance is labeled with the above inorganic fluorescent nanoparticles, a functional group needs to be introduced which bonds to both the particles and the living substance. For this, the following manner was employed.

<Introduction of a Modifying Functional Group into Si/SiO₂.Core/Shell Particles>

By use of bonding of mercapto groups (SH groups), a carboxyl group is introduced into fluorescent semiconductor fine particles.

Initially, the above Si core/shell particles are dispersed in a 30% hydrogen peroxide solution for 10 minutes to hydroxylate the crystal surface. Then, the solvent is replaced with toluene and mercaptopropyltriethoxysilane is added at 2% of the toluene. SiO₂ on the uppermost surface of the Si core particles is silanized and simultaneously a mercapto group is introduced over 2 hours. Subsequently, the solvent was replaced with water and a buffer salt was added. Further, a compound having a mercapto group introduced into one terminal thereof, listed in Table 1, was selected and added at an appropriate amount, followed by stirring for 3 hours. Then, the Si core particles are allowed to bond to the selected acid. In Table 1, the selected surface modifying compounds and the length ratio thereof to each of the corresponding nanoparticle sizes (Labeling Material Size Ratio) were listed.

Herein, main surface modifying compounds determining the size were listed in Table 1. In fine adjustment for the size of the present invention, size adjustment is carried out via utilization of ester bonding to an alcohol.

(Introduction of a Modifying Functional Group into Si/ZnS.Core/Shell Particles)

The above-obtained Si/ZnS.core/shell particles were dispersed in a buffer salt solution and an acid of the same kind as described above was added at an appropriate amount, followed by stirring at an appropriate temperature for 2 hours to allow the particle surface to bond to a mercapto group. In this manner, each of the surface modifying compounds with a carboxyl group featuring the length ratio listed in Table 1 is introduced into the surface.

With regard to specific gravity in the cases of the presence and absence of a surface modifying compound, evaluation was made using those produced above and the results were listed in Table 1 as Labeling Material Specific Gravity Ratio.

Specific gravity was determined using SMK-301 (produced by Shimadzu Corp.) based on its manual.

The above-obtained labeling material was previously mixed with sheep serum albumin (SSA) at the same concentration and individually allowed to be introduced into Vero cells. After culture at 37° C. for 2 hours, trypsinization was carried out for resuspension in 5% FBS added DMEM, followed by sowing in the same glass bottom dish. Cells having been cultured at 37° C. overnight were fixed with 4% formalin and the nuclei were stained with DAPI, followed by infrared fluorescence observation using a confocal laser scanning microscope (excitation at 700 nm).

The state of accumulation into a membrane protein in the case of introduction of the present labeling material into a cytoplasmic endosome was evaluated by the concentration and the dispersion state which depend on fluorescence intensity. Namely, when the present labeling material is introduced into a cell and the migration efficiency of migration and accumulation into an endosome is uniform and larger fluorescence intensity in the endosome is increased and also its distribution is uniform and wide. This result reflects the situation with no aggregation or association of the labeling material. On the other hand, when introduction and migration efficiency decrease due to effects such as aggregation and specific gravity, fluorescence intensity is small and non-uniform mottled patterns are generated, whereby emission intensity differs to a large extent at locations and also a small emission accumulation area results. The condition of this observation was described in Table 1.

TABLE 1

| | | | | | | Labeling | | |
|---|---|---|---|---|---|---|---|---|
| | | | Inorganic | | Labeling | Material | | |
| | Core | | Fluorescent | Surface | Material | Specific | | |
| | Production | | Nanoparticle | Modifying | Size | Gravity | Detection Performance | |
| No. | Method | Shell | (nm) | Compound | Ratio | Ratio | Observation | Remarks |
| 1 | HF METHOD | SiO₂ | 3.0 | D | 0.2 | 0.6 | high and uniform detection | inventive |
| | | | | | | | sensitivity | |
| 2 | anodization | SiO₂ | 4.8 | G | 0.35 | 0.55 | uniform and high sensitivity | inventive |
| 3 | HF METHOD | ZnS | 3.0 | C | 0.2 | 0.6 | uniform and high sensitivity | inventive |
| 4 | anodization | ZnS | 4.8 | H | 0.35 | 0.55 | uniform and high sensitivity | inventive |
| 5 | HF METHOD | SiO₂ | 3.0 | A | 0.1 | 0.95 | non-uniform and medium | comparative |
| | | | | | | | sensitivity | |
| 6 | anodization | SiO₂ | 4.8 | C | 0.15 | 0.9 | non-uniform and medium | comparative |
| | | | | | | | sensitivity | |
| 7 | HF METHOD | SiO₂ | 3.0 | G | 1.0 | 0.8 | slightly non-uniform and low | comparative |
| | | | | | | | sensitivity | |
| 8 | anodization | SiO₂ | 4.8 | *1 | 0.8 | 0.8 | slightly non-uniform and low | comparative |
| | | | | | | | sensitivity | |
| 9 | HF METHOD | SiO₂ | 3.0 | *2 | 1 | 0.95 | extremely low sensitivity | ** |
| | | | | | | | and scattering | |
| 10 | HF METHOD | SiO₂ | 25.2 | *3 | 0.35 | 0.55 | detection almost impossible | comparative |
| 11 | HF METHOD | SiO₂ | 25.2 | *4 | 1.0 | 0.95 | detection impossible | comparative |

*1 Used were compound G and a synthesis reaction product of polyethylene glycol of a carbon atom number realizing a specified labeling material size ratio and adipic acid
*2 Used were compound F and a synthesis reaction product of polyethylene glycol of a carbon atom number realizing a specified labeling material size ratio and adipic acid
*3 Used were compound F and a synthesis reaction product of polyethylene glycol of a carbon atom number realizing a specified labeling material size ratio and adipic acid
*4 Used were compound F and a synthesis reaction product of polyethylene glycol of a carbon atom number realizing a specified labeling material size ratio and adipic acid
** failed modification (comparative)

In Table 1, Labeling Material Size Ratio refers to “a ratio of the length of a surface modifying compound from the surface of an inorganic fluorescent nanoparticle to the particle diameter of the inorganic fluorescent nanoparticle.” Labeling Material Specific Gravity Ratio refers to “a ratio of the specific gravity of an inorganic fluorescent nanoparticle bonded with a surface modifying compound to the specific gravity of an inorganic fluorescent nanoparticle bonded with no surface modifying compound.”

As shown in Table 1, any labeling fluorescent compound according to the constitution of the present invention is proved to exhibit extremely stable detection performance in vital labeling with high sensitivity.

1. A labeling fluorescent compound comprising an inorganic fluorescent nanoparticle which is provided with a surface modifying compound on a surface of the inorganic fluorescent nanoparticle, provide that the inorganic fluorescent nanoparticle has an average particle diameter of 1.0-20 nm,
wherein a ratio of a length of the surface modifying compound from the surface of the inorganic fluorescent nanoparticle to a particle diameter of the inorganic fluorescent nanoparticle is 0.10-0.50; and
a ratio of a specific gravity of the inorganic fluorescent nanoparticle bonded with the surface modifying compound to a specific gravity of an inorganic fluorescent nanoparticle non-bonded with the surface modifying compound is 0.80-0.40.

2. The labeling fluorescent compound described in claim 1,
wherein the inorganic fluorescent nanoparticle has a core/shell structure composed of a semiconductor nanocrystal core and a semiconductor shell.

3. The labeling fluorescent compound described in claim 1,
wherein the inorganic fluorescent nanoparticle emits infrared radiation by excitation with rays in the range of near-infrared radiation to infrared radiation.

4. The labeling fluorescent compound described in claim 2,
wherein the inorganic fluorescent nanoparticle emits infrared radiation by excitation with rays in the range of near infrared radiation to infrared radiation.
